(19) United States
(12) Patent Application Publication
(10) Pub. No.: US 2019/0159659 A1
(43) Pub. Date: May 30, 2019

(54) ENDOSCOPIC SYSTEM, OVER-TUBE, AND CAP

(71) Applicant: Olympus Corporation, Tokyo (JP)

(72) Inventors: Jun Wakasone, Wako-shi (JP); Takumi Isoda, Tokyo (JP)

(73) Assignee: Olympus Corporation, Tokyo (JP)

(21) Appl. No.: 16/263,622

(22) Filed: Jan. 31, 2019

Related U.S. Application Data
(63) Continuation of application No. PCT/JP2016/086132, filed on Dec. 6, 2016.

(51) Int. Cl. A61B 1/00; A61B 1/018; A61B 1/05

(52) U.S. Cl. CPC A61B 1/00135; A61B 1/00087; A61B 1/01; A61B 1/05; A61B 1/00154; A61B 1/018

(57) ABSTRACT

The technology disclosed herein is directed to an over-tube, an endoscopic system, and a cap which is constructed to identify with greater certainty from which position a medical instrument inserted in a channel will project on the distal end of the over-tube. The over-tube includes a tubular member and a protrusion. The tubular member includes at least a treatment tool channel into which a treatment tool is inserted and an endoscope channel into which an endoscope device is inserted. The protrusion protrudes radially inwardly from at least a portion of an inner circumferential surface of the endoscope channel in a longitudinal direction thereof. The protrusion indicates a position configuration in a circumferential direction of the treatment tool channel with respect to the endoscope channel. The endoscope captures an image that includes the protrusion and the protrusion in the acquired image confirms the position configuration of the treatment tool channel.

### CROSS-REFERENCE TO RELATED APPLICATION

This application is a continuation application of PCT Application No. PCT/JP2016/086132 filed on Dec. 6, 2016, which is hereby incorporated by reference in its entirety.

### TECHNICAL FIELD

The technology disclosed herein relates generally to an endoscopic system, and more particularly, some embodiments relate to a combination of an over-tube, an endoscopic system, and a cap.

### DESCRIPTION OF THE RELATED ART

A Japanese Patent Application JP 2013-172780A discloses an over-tube having an identifier formed by radially outwardly denting a portion of the inner circumferential surface of a channel in the over-tube. The identifier is visually recognized with a medical instrument inserted in the channel. A user can recognize from which position the medical instrument is inserted in the channel so that it projects on the distal end of the over-tube.

However, when the identifier is formed by denting a portion of the inner surface of the channel, the identifier is often difficult to visually recognize with an observational instrument inserted in the channel. In particular, if a body fluid or the like is deposited in the channel, then the dented identifier tends to be filled up with the body fluid or the like, and hence the dented identifier becomes more difficult to be visually recognized.

Therefore, there is a need for an over-tube, an endoscopic system, and a cap that overcome the aforementioned shortcomings in the prior art.

### BRIEF SUMMARY OF EMBODIMENTS

Embodiments of the technology disclosed herein is directed to an elongated over-tube, an endoscopic system, and a cap which is constructed to identify with greater certainty from which position a medical instrument inserted in a channel will project on the distal end of the elongated over-tube.

According to one embodiment of the technology disclosed herein, an elongated over-tube includes an elongated tubular member and a protrusion. The elongated tubular member includes a treatment tool channel into which a treatment tool can be inserted and an endoscope channel into which an endoscope can be inserted. The protrusion protrudes radially inwardly from at least a portion of an inner circumferential surface of the endoscope channel in a longitudinal direction thereof. The protrusion indicates a direction that the treatment tool channel exists with respect to the endoscope channel.

With the above embodiment, an endoscope is inserted into the endoscope channel of the tubular member, and then captures an image of an inner surface of the endoscope channel. The endoscope captures an image that includes the protrusion that protrudes radially inwardly from at least the portion of the inner circumferential surface of the endoscope channel in the longitudinal direction thereof. The protrusion in the acquired image makes it possible for the user to confirm the position configuration of the treatment tool channel in a circumferential direction with respect to the endoscope channel. In other words, the protrusion lets the user know in what position configuration the treatment tool will appear in an endoscopic image before the treatment tool projects from the distal end of the tubular member through the treatment tool channel and appears in the endoscopic image. The protrusion may be disposed on a straight line interconnecting the center of the endoscope channel and the center of the treatment tool channel. The protrusion makes it easy for the user to know in the endoscopic image that the treatment tool channel exists on an extension of a straight line interconnecting the center of the image and the protrusion in the image. The protrusion may be disposed in the vicinity of the distal end of the endoscope channel in the longitudinal direction thereof.

With this arrangement, even if the over-tube is twisted and the treatment tool channel is in different positions with respect to the endoscope channel at the proximal end and the distal end of the over-tube, the user can confirm the position of the treatment tool channel in the circumferential direction by the protrusion. The protrusion is disposed in the vicinity of the distal end and the endoscope will project from the distal end. Therefore, the practitioner would know properly in which position configuration the treatment tool will appear in the endoscopic image. The protrusion may be elastically deformable radially outwardly by being pushed by the endoscope inserted in the endoscope channel. When the endoscope is apart from the protrusion, the protrusion protrudes radially inwardly to a large extent, increasing its visibility in the endoscopic image. After the endoscope is contacted the protrusion, the protrusion is elastically deformed radially outwardly by the endoscope, increasing the cross-sectional area of a passage through which the endoscope passes thereby to prevent the insertability of the endoscope from being lowered. The protrusion may have a slope which protrudes radially inwardly by a distance that progressively varies from the proximal end toward the distal end of the endoscope channel. Although the protrusion reduces the cross-sectional area of a passage for endoscope to travel, the slope of the protrusion can guide the endoscope as it moves from the proximal end toward the distal end of the over-tube to go easily over the protrusion, thereby preventing the insertability of the endoscope from being lowered. The protrusion may have a shape which is different for each treatment tool channel. With this arrangement, it is possible to identify the type of the treatment tool channel by confirming the shape of the protrusion in the endoscopic image acquired by the endoscope.

According to another embodiment of the technology disclosed herein, a cap is mounted on the distal end of an elongated over-tube. The elongated over-tube includes a tubular member having a treatment tool channel into which a treatment tool is inserted and an endoscope channel into which an endoscope is inserted. The cap includes a treatment tool exit hole, an endoscope exit hole, and a protrusion. The treatment tool exit hole is defined at a position that is held in fluid communication with the treatment tool channel. The endoscope exit hole is defined at a position that is held in fluid communication with the endoscope channel. The protrusion protrudes radially inwardly from an inner circumferential surface of the endoscope exit hole. The protrusion indicates a circumferential position of the treatment tool exit hole with respect to the endoscope exit hole. The cap is mounted on the distal end of the over-tube. The endoscope channel in the over-tube and the endoscope exit hole in the cap are held in fluid communication with each other. The treatment tool channel in the over-tube and the treatment tool exit hole in the cap are held in fluid communication with each other. When an endoscope is placed in the endoscope channel and captures an endoscopic image, the endoscopic image includes the protrusion that protrudes radially inwardly from the inner circumferential surface of the endoscope exit hole. The protrusion in the endoscopic image lets the user know in which position the treatment tool will appear in the endoscopic image before the treatment tool projects from the treatment tool exit hole through the treatment tool channel and appears in the endoscopic image.

According to still further embodiment of the technology disclosed herein, an endoscopic system includes any one of the described-herein over-tubes, an endoscope that is inserted in the endoscope channel in the over-tube, and the treatment tool that is inserted in the treatment tool channel in the over-tube. The technology disclosed herein offers the advantages such as to identify with greater certainty from which position a medical instrument inserted in a channel will project on the distal end of an elongated over-tube.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a perspective view of a portion of an endoscopic system illustrating a distal end portion thereof according to an embodiment of the technology disclosed herein.

FIG. 2 is an end view of a portion of an elongated over-tube depicted in FIG. 1 when the endoscope device and the treatment tools are removed.

FIG. 3 is a fragmentary longitudinal cross-sectional view taking along line 3-3 of the elongated over-tube depicted in FIG. 2.

FIG. 4 is a diagram depicting by way of example an image of the inside of an endoscope channel defined in the over-tube depicted in FIG. 2, which image is acquired by an endoscope inserted in the endoscope channel.

FIG. 5 is the same as the elongated over-tube depicted in FIG. 2, with the endoscope inserted in the endoscope channel in the over-tube.

FIG. 6 is a fragmentary longitudinal cross-sectional view depicting a first modification of the over-tube depicted in FIG. 3.

FIG. 7 is a fragmentary longitudinal cross-sectional view depicting a second modification of the over-tube depicted in FIG. 3.

FIG. 8A is similar to FIG. 2 depicting a third modification of the over-tube.

FIG. 8B is of the same as the over-tube depicted in FIG. 8A, with the endoscope inserted in the endoscope channel in the over-tube.

FIG. 9 is similar to FIG. 2 depicting a fourth modification of the over-tube.

FIG. 10 is similar to FIG. 2 depicting a fifth modification of the over-tube depicted.

FIG. 11 is a longitudinal cross-sectional view taking along line 11-11 depicting a cap, which is mounted on the distal end of the over-tube.

FIG. 12 is a perspective view of a cap mounted on the distal end of the over-tube.

FIG. 13 is a perspective view depicting a modification of the cap depicted in FIG. 11.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

An over-tube 1 and an endoscopic system 100 according to one embodiment of the technology disclosed herein are described hereinafter with reference to the drawings.

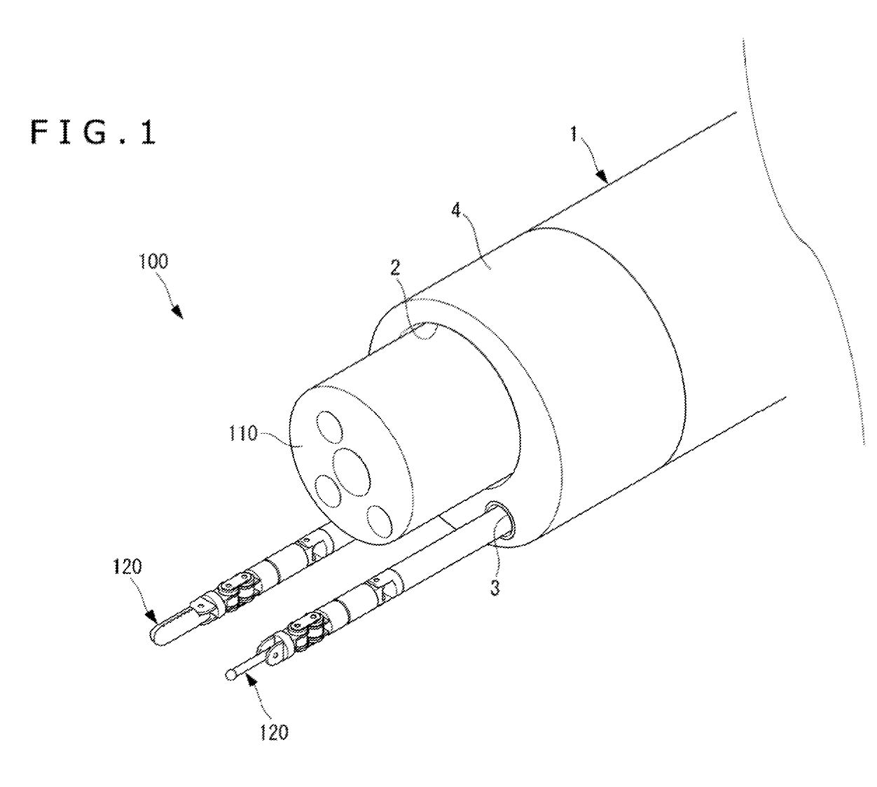

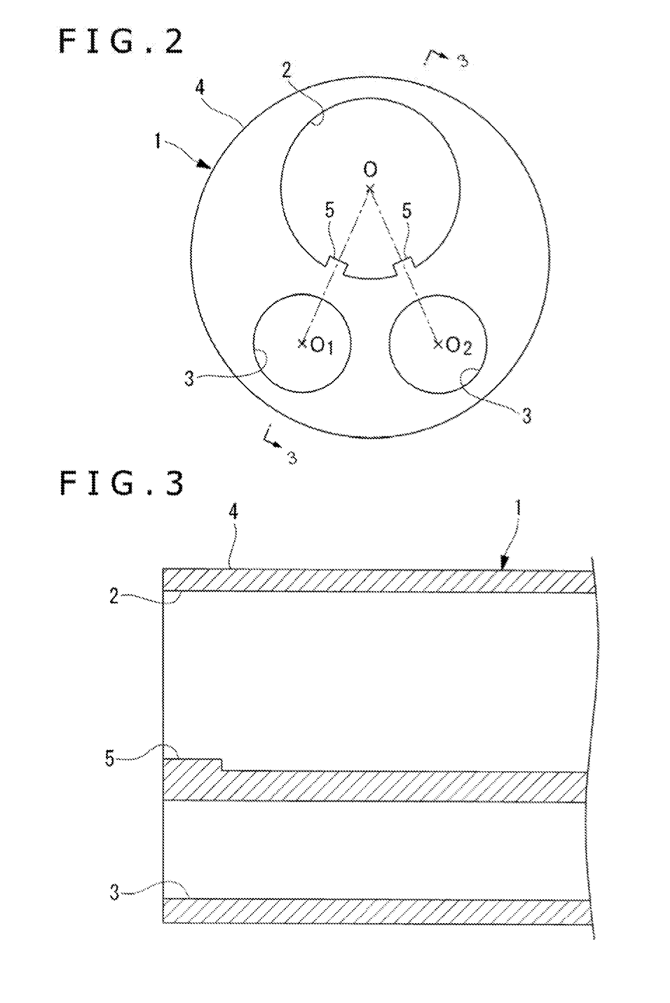

As depicted in FIG. 1, the endoscopic system 100 includes an endoscope 110, two treatment tools 120, and an elongated over-tube 1. The elongated over-tube 1 includes an elongated tubular member 4. The elongated tubular member 4 is preferably made of a flexible material and includes an endoscope channel 2 and two treatment tool channels 3 along a longitudinal direction thereof. The endoscope 110 is inserted in the endoscope channel 2. Respective treatment tools 120 are inserted into respective treatment tool channels 3. As depicted in FIG. 2, the respective endoscope channel 2 and the two treatment tool channels 3 in the tubular member 4 are of a substantial circular cross-sectional shape.

As depicted in FIGS. 2 and 3, protrusions 5 that protrude radially inwardly are provided on an inner circumferential surface of the endoscope channel 2 at distal-end positions in the tubular member 4. “Protruding radially inwardly” means protruding from the inner surface of the endoscope channel 2 toward the center (o) of the endoscope channel 2 in FIG. 2. As depicted in FIG. 2, the respective protrusions 5 are disposed on respective straight lines interconnecting the center (O) of the endoscope channel 2 and the centers (O₁) and (O₂) of the two treatment tool channels 3. Each of the protrusions 5 has a rectangular cross-sectional shape. The protrusion 5 extends a predetermined length from the distal end of the elongated tubular member 4 along the longitudinal direction of the endoscope channel 2 as seen best in FIG. 3. Operation of the elongated over-tube 1 and the endoscopic system 100 thus constructed according to the present embodiment is now described.

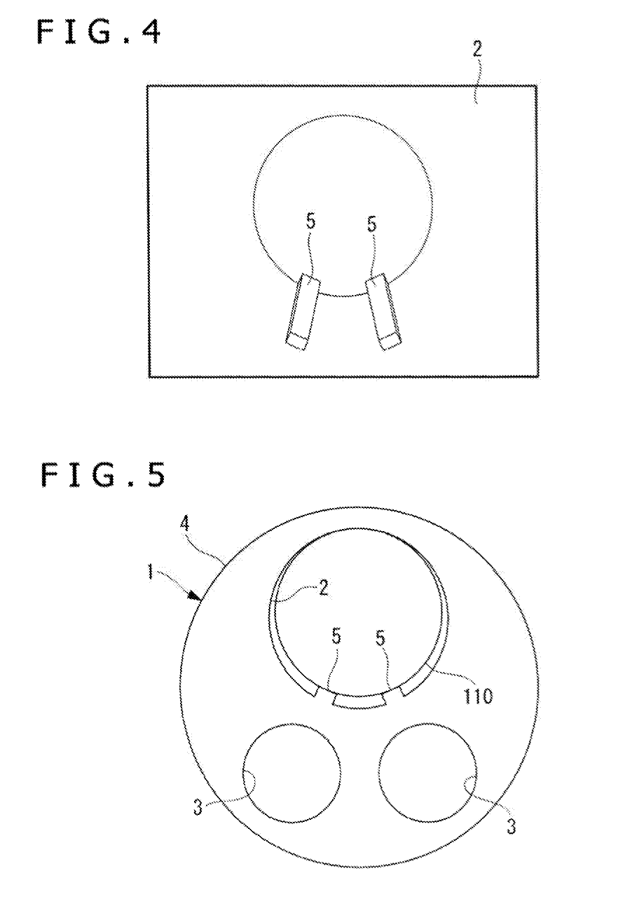

The endoscope 110 is inserted into the endoscope channel 2 in the elongated over-tube 1 according to the present embodiment. When the endoscope 110 is energized, it acquires an image of an inner surface of the endoscope channel 2. The endoscope 110 is moved forwardly in the endoscope channel 2. When the distal end of the endoscope 110 comes near the opening of the endoscope channel 2, the endoscope 110 captures an image of the protrusions 5 provided at the distal end of the endoscope channel 2 and acquires an endoscopic image as depicted in FIG. 4. The protrusions 5 are disposed on the respective straight lines interconnecting the center (O) of the endoscope channel 2 and the centers (O₁) and (O₂) of the treatment tool channels 3 as depicted in FIG. 2. Therefore, the user can easily confirm that the treatment tool channels 3 exist in the direction of the protrusions 5 in the endoscopic image depicted in FIG. 4. In other words, with the elongated over-tube 1 and the endoscopic system 100 in operation, even though the action of (i) projecting the treatment tools 120 from the distal end of the over-tube 1 and (ii) directly capturing an image of the treatment tools 120 with the endoscope 110 as depicted in FIG. 1 is not carried out, the user can learn in advance in which positions the treatment tools 120 appear in the image acquired by the endoscope 110. While the elongated over-tube 1 and the endoscope 110 are being inserted along a tortuous path such as tortuous colon or a body cavity in a patient, the phase of the endoscope 110 and the over-tube 1 about their longitudinal axes may change due to a twist in the over-tube 1. Even in such a situation, the user can easily recognize the locations where the respective treatment tools 120 will project from the over-tube 1 by confirming the positions where the treatment tools 120 appear in the image before the respective treatment tools 120 project from the distal end of the over-tube 1.

According to the present embodiment, furthermore, to the extent that the protrusions 5 are provided in the vicinity of the distal end of the endoscope channel 2, the protrusions 5 can project into part of the profile of the exit of the endoscope channel 2 in the endoscopic image. Specifically, the protrusions 5 in the endoscopic image can be recognized not only as luminance changes based on shape changes from the surrounding wall surface, but also as profile shapes of the exit of the endoscope channel 2. As a result, the visibility of the protrusions 5 is prevented from being lowered due to their assimilating into the surrounding wall surface, allowing the user to recognize the positions of the protrusions 5 more reliably than if the protrusions 5 are disposed at positions apart from the distal end of the endoscope channel 2 toward the proximal end thereof. Moreover, when the endoscope 110 is placed at the distal end of the endoscope channel 2, the endoscope 110 is disposed between radially inner surfaces of the protrusions 5 and an inner circumferential surface of the endoscope channel 2 that is opposite those radially inner surfaces. Gaps are created between the endoscope 110 and the inner circumferential surface of the endoscope channel 2 except for the protrusions 5 as depicted in FIG. 5. As a consequence, a body fluid or the like that may have entered the endoscope channel 2 tends to flow through the gaps, rather than staying in the endoscope channel 2, so that the endoscope channel 2 is kept in a state that makes the protrusions 5 visually recognizable.

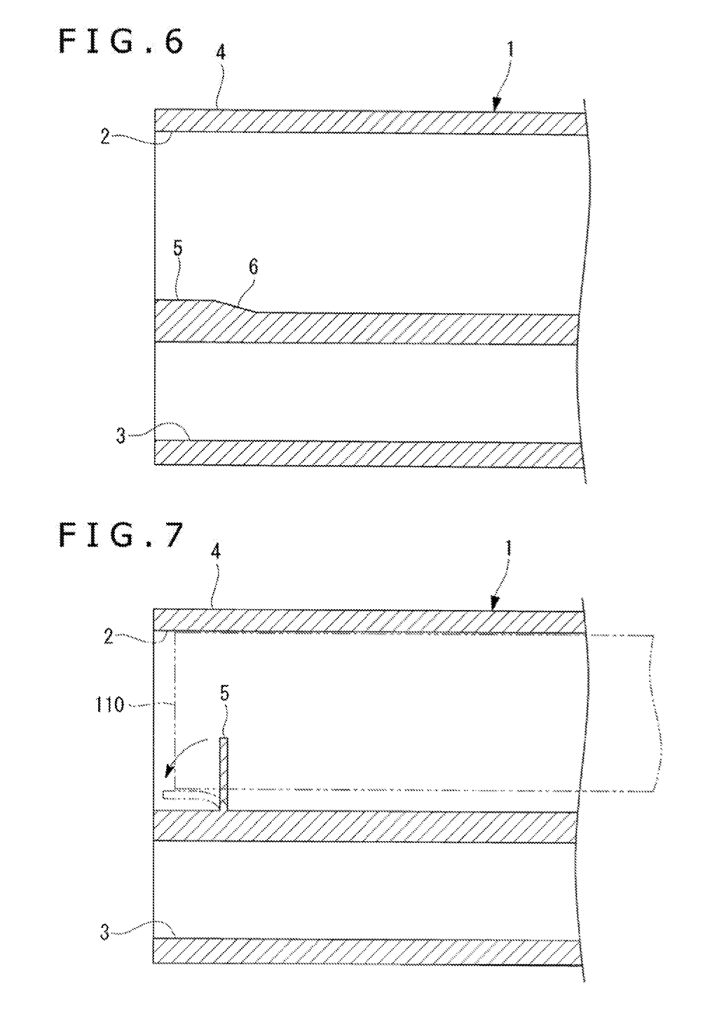

According to the present embodiment, each of the protrusions 5 is in the form of a rectangular parallelepiped having a constant cross-sectional shape. However, as depicted in FIG. 6, a protrusion 5 may have a slope 6 on a proximal end portion thereof, whose height is progressively larger toward the distal end thereof. The slope 6 of the protrusion 5 improves the insertability of the endoscope 110 because the distal end of the endoscope 110 is moving forwardly in the endoscope channel 2 and the distal end is not caught by any step on the proximal end of the protrusion 5. Specifically, the edge of the distal-end face of the endoscope 110 rides onto the slope 6 and moves over the protrusion 5 without being caught thereby, so that the endoscope 110 can easily project out of the distal end of the elongated tubular member 4. The protrusions 5 attached to the inner circumferential surface of the endoscope channel 2 are illustrated. Instead, as depicted in FIG. 7, a protrusion 5 may be formed integrally with the elongated tubular member 4. The protrusion 5 is preferably made of an elastic material such as silicone resin. In this case, the protrusion 5 may be elastically deformable from a solid-line state into a chain-line state as indicated by the arrow in FIG. 7 when the protrusion 5 is pushed by the distal-end face of the endoscope 110 that has been moving forwardly in the endoscope channel 2.

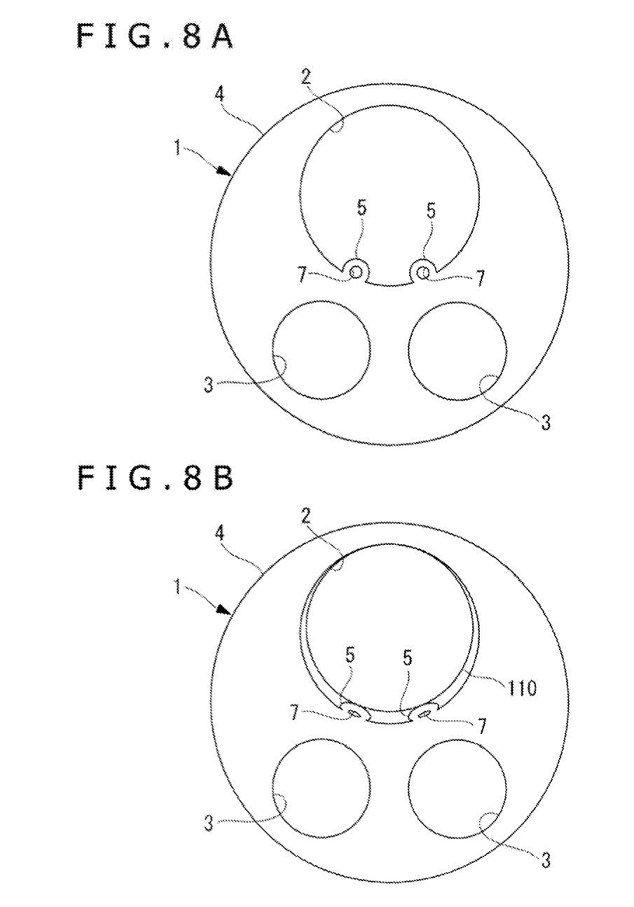

With the protrusion 5 being elastically deformable, the height of the protrusion 5 from the inner circumferential surface of the endoscope channel 2 is rendered variable. Until the endoscope 110 reaches the protrusion 5, the protrusion 5 protrudes to a large extent, increasing its visibility in the endoscopic image. After the endoscope 110 reached the protrusion 5, the projecting height thereof is then reduced, which in turn, increasing the cross-sectional area of a passage and it is easy for the endoscope 110 to pass through the passage. Furthermore, as depicted in FIG. 8A, each protrusion 5 may be formed integrally with the elongated tubular member 4 and may be of a hollow structure having a cavity 7 therein. Each of the protrusion 5 is made of an elastic material such as silicone resin. In this case, as depicted in FIG. 8B, each protrusion 5 may be of such a structure that it can easily collapse, i.e., it can be elastically deformed radially outwardly, by being pushed by the endoscope 110, reducing its projecting height. Each protrusion 5 is of a rectangular cross-sectional shape. Instead, each protrusion 5 may have any of other optional cross-sectional shapes. Alternatively, the protrusions 5 that are provided in association with the respective treatment tool channels 3 may be of different cross-sectional shapes. With the protrusions 5 having different cross-sectional shapes, even if one of the protrusions 5 is hidden by a tissue or the like or is dislodged, the shape of the other protrusion 5 helps the user to recognize with which one of the treatment tool channels 3 the hidden or dislodged protrusion 5 is associated.

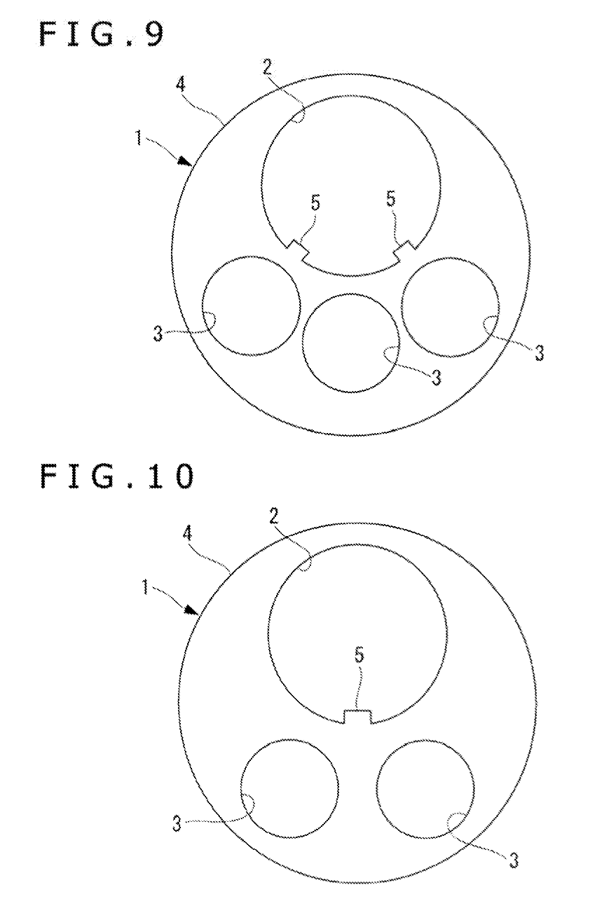

According to the present embodiment, the protrusions 5 are disposed on the respective straight lines interconnecting the center (O) of the endoscope channel 2 and the centers (O₁) and (O₂) of the treatment tool channels 3. Rather, the protrusions 5 may not strictly be on the straight lines, but may be positioned off the straight lines. The protrusions 5 are provided in association with the respective treatment tool channels 3 in the present embodiment. When one or more treatment tool channels 3 (three in FIG. 9) are arrayed on one side of the endoscope channel 2 as depicted in FIG. 9, for example, the protrusions 5 may be provided on both sides of the range of the array of the treatment tool channels 3. Furthermore, as depicted in FIG. 10, in case of where two treatment tool channels 3 are arranged side by side, a single protrusion 5 may be provided in the vicinity of a central region therebetween. In other words, the number and positions of protrusions 5 may be arbitrary to the extent that they are able to indicate the positions of treatment tool channels 3 with respect to the endoscope channel 2 in the endoscopic image. The protrusions 5 are illustrated as they are disposed at the distal end of the endoscope channel 2. However, such a layout is not restrictive or limited, but the protrusions 5 may be provided in any positions in the longitudinal direction of the tubular member 4, or may be provided anywhere closer to the distal end from the central position in the longitudinal direction of the tubular member 4. Though it is particularly preferable for the protrusions 5 to be disposed at the distal end of the endoscope channel 2, they may be disposed at positions slightly apart from the distal end toward the proximal end. Moreover, the protrusions 5 are illustrated as being integrally provided in the endoscope channel 2 in the elongated tubular member 4 of the elongated over-tube 1.

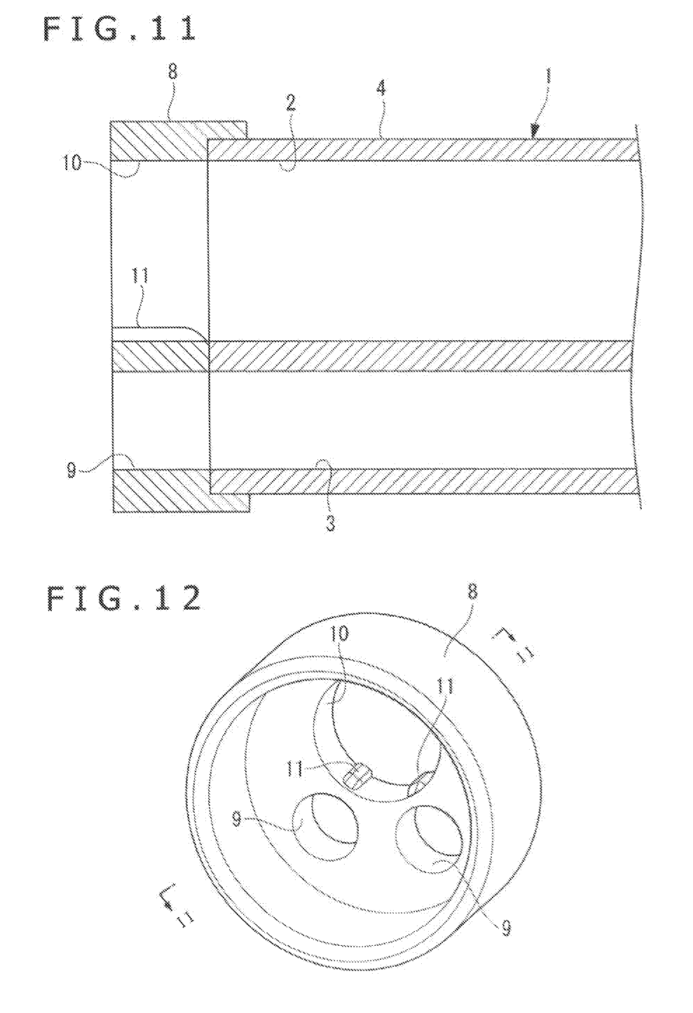

Instead, as depicted in FIGS. 11 and 12, there may be employed a cap 8 mounted on the distal end of a tubular member 4 which has a uniform cross-sectional shape. The cap 8 is attached to the distal end of the tubular member 4 by an adhesive or the like, or may be detachably mounted thereon. The cap 8 has treatment tool exit holes 9, an endoscope exit hole 10, and protrusions 11. The treatment tool exits holes 9 are defined at positions that are held in fluid communication with the treatment tool channels 3 when the cap 8 is mounted on the distal end of the tubular member 4. The endoscope exit hole 10 is defined at a position that is held in fluid communication with the endoscope channel 2 when the cap 8 is mounted on the distal end of the tubular member 4. The protrusions 11 protrude radially inwardly from an inner circumferential surface of the endoscope exit hole 10, for indicating circumferential positions of the treatment tool exit holes 9 with respect to the endoscope exit hole 10. The protrusions 11 are disposed on respective straight lines interconnecting the center of the endoscope exit hole 10 and the centers of the treatment tool exit holes 9. Simply by mounting the cap 8 on the distal end of the tubular member 4, the directions of the treatment tool channels 3 can be indicated from the positions of the protrusions 11 in the endoscopic image captured by the endoscope 110 that is placed in the endoscope channel 2.

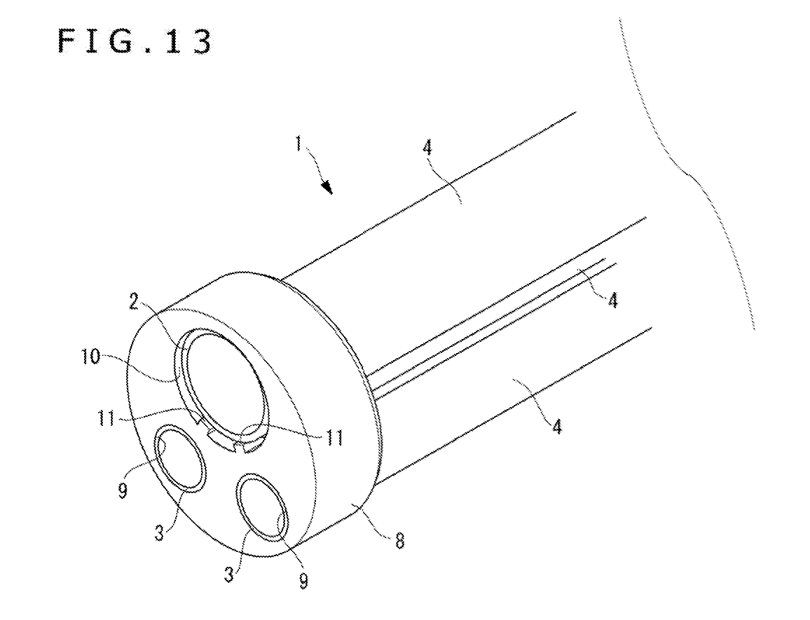

The cap 8 according to the present embodiment is mounted on the elongated tubular member 4 that is provided as a single member which has the endoscope channel 2 and the treatment tool channels 3. Alternatively, as depicted in FIG. 13, the cap 8 may be mounted on a tubular member 4 that is made up of three members which separately have the endoscope channel 2 and the two treatment tool channels 3. Furthermore, the elongated tubular member 4 may be made up of two members.

In sum, the disclosed technology is directed to an over-tube comprises an elongated tubular member having a treatment tool channel into which a treatment tool is inserted and an endoscope channel into which an endoscope is inserted. A protrusion is configured to protrude radially inwardly from at least a portion of an inner circumferential surface of the endoscope channel in a longitudinal direction thereof. The protrusion is configured to indicate a direction that the treatment tool channel exits with respect to the endoscope channel.

The protrusion is disposed on a straight line interconnecting a center of the endoscope channel and a center of the treatment tool channel to one another. The protrusion is disposed in a vicinity of a distal end of the endoscope channel in the longitudinal direction thereof. The protrusion is elastically deformable radially outwardly by being pushed by the endoscope inserted in the endoscope channel. The protrusion has a slope which protrudes radially inwardly by a distance that progressively varies from a proximal end toward the distal end of the endoscope channel. The treatment tool channel has a first treatment tool channel and a second treatment tool channel. The protrusion has a first protrusion and a second protrusion and has a shape which is different for each treatment tool channel.

Another aspect of the disclosed technology is directed to a cap is mounted on a distal end of an over-tube that includes a tubular member having a treatment tool channel into which a treatment tool can be inserted and an endoscope channel into which an endoscope can be inserted. The cap comprises a treatment tool exit hole disposed at a position that is held in fluid communication with the treatment tool channel. An endoscope exit hole is disposed at a position that is held in fluid communication with the endoscope channel. A protrusion is configured to protrude radially inwardly from an inner circumferential surface of the endoscope exit hole and configured to indicate a circumferential position of the treatment tool exit hole with respect to the endoscope exit hole.

A further aspect of the disclosed technology is directed to an endoscopic system comprises an elongated over-tube having at least one treatment tool channel and an endoscope channel spaced apart from one another and is longitudinally formed therein, respectively. At least one treatment tool is configured to be inserted into the treatment tool channel. An endoscope is configured to be inserted into the endoscope channel. The endoscope channel includes a protrusion formed in a vicinity of a distal end of the endoscope channel in a longitudinal direction and is configured to indicate a position in a circumferential direction of the treatment tool channel with respect to the endoscope channel. The protrusion is configured to protrude radially inwardly from at least a portion of an inner circumferential surface of the endoscope channel in the longitudinal direction. The at least one treatment tool is defined by respective first and second treatment tools spaced apart from one another and being longitudinally formed therein.

Yet another aspect of the disclosed technology is directed to an endoscopic system having an elongated over-tube used therein. The elongated over-tube comprises an elongated tubular member being defined by two treatment channel tools and an endoscope channel all of which are spaced apart from one another and are longitudinally formed therein so as to receive respective treatment tools and an endoscope for operation in a body of a patient. The endoscope channel includes a protrusion formed in a vicinity of a distal end of the endoscope channel in the longitudinal direction and is configured to indicate a position in a circumferential direction of the treatment tool channel with respect to the endoscope channel. The endoscopic system further comprises a cap being mounted on a distal end of an over-tube. the protrusion is disposed on a straight line interconnecting a center of the endoscope channel and a center of the treatment tool channel to one another.

The protrusion has a slope which protrudes radially inwardly by a distance that progressively varies from a proximal end toward the distal end of the endoscope channel. When a distal end of the endoscope comes near an opening of the endoscope channel, the endoscope captures an image of the protrusion provided at the distal end of the endoscope channel and acquires an endoscopic image. The protrusion in the endoscopic image permits a user to know in which position the treatment tool will appear in the endoscopic image before the treatment tool projects from the treatment tool exit hole through the treatment tool channel and appears in the endoscopic image. Gaps are formed between the endoscope and an inner circumferential surface of the endoscope channel except for the protrusion so that a body fluid that being entered the endoscope channel tends to flow through the gaps rather than staying in the endoscope channel so that the endoscope channel is kept in a state that makes the protrusion visually recognizable.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one”, “one or more” or the like; and adjectives such as “conventional”, “traditional”, “normal”, “standard”, “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more”, “at least”, “but not limited to” or other like phrases in some instances shall not be read to mean that the narrower case is intended or required in instances where such broadening phrases may be absent.

Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. An over-tube comprising:
an elongated tubular member having a treatment tool channel into which a treatment tool being inserted and an endoscope channel into which an endoscope being inserted; and
a protrusion configured to protrude radially inwardly from at least a portion of an inner circumferential surface of the endoscope channel in a longitudinal direction thereof, and configured to indicate a direction that the treatment tool channel exits with respect to the endoscope channel.

2. The over-tube of claim 1, wherein the protrusion is disposed on a straight line interconnecting a center of the endoscope channel and a center of the treatment tool channel to one another.

3. The over-tube of claim 1, wherein the protrusion is disposed in a vicinity of a distal end of the endoscope channel in the longitudinal direction thereof.

4. The over-tube of claim 1, wherein the protrusion is elastically deformable radially outwardly by being pushed by the endoscope inserted in the endoscope channel.

5. The over-tube of claim 1, wherein the protrusion has a slope which protrudes radially inwardly by a distance that progressively varies from a proximal end toward the distal end of the endoscope channel.

6. The over-tube of claim 1, wherein
the treatment tool channel has a first treatment tool channel and a second treatment tool channel,
the protrusion has a first protrusion and a second protrusion, and
the protrusion has a shape which is different for each treatment tool channel.

7. A cap being mounted on a distal end of an over-tube that includes a tubular member having a treatment tool channel into which a treatment tool can be inserted and an endoscope channel into which an endoscope can be inserted, the cap comprising:
a treatment tool exit hole disposed at a position that is held in fluid communication with the treatment tool channel;
an endoscope exit hole disposed at a position that is held in fluid communication with the endoscope channel; and
a protrusion configured to protrude radially inwardly from an inner circumferential surface of the endoscope exit hole and configured to indicate a circumferential position of the treatment tool exit hole with respect to the endoscope exit hole.

8. An endoscopic system comprising:
an elongated over-tube having at least one treatment tool channel and an endoscope channel spaced apart from one another and being longitudinally formed therein, respectively;
at least one treatment tool configured to be inserted into the treatment tool channel; and
an endoscope configured to be inserted into the endoscope channel wherein the endoscope channel includes a protrusion formed in a vicinity of a distal end of the endoscope channel in a longitudinal direction and being configured to indicate a position in a circumferential direction of the treatment tool channel with respect to the endoscope channel.

9. The endoscopic system of claim 8, wherein the protrusion being configured to protrude radially inwardly from at least a portion of an inner circumferential surface of the endoscope channel in the longitudinal direction.

10. The endoscopic system of claim 8, wherein the protrusion has a slope that protrudes radially inwardly by a distance that progressively varies from a proximal end toward the distal end of the endoscope channel.

11. The endoscopic system of claim 8, wherein the at least one treatment tool is defined by respective first and second treatment tools spaced apart from one another and being longitudinally formed therein.

12. An endoscopic system having an elongated over-tube used therein, the elongated over-tube comprising:
an elongated tubular member being defined by two treatment channel tools and an endoscope channel all of which being spaced apart from one another and being longitudinally formed therein so as to receive respective treatment tools and an endoscope for operation in a body of a patient wherein
the endoscope channel includes a protrusion formed in a vicinity of a distal end of the endoscope channel in the longitudinal direction and being configured to indicate a position in a circumferential direction of the treatment tool channel with respect to the endoscope channel.

13. The endoscopic system of claim 12, further comprising a cap being mounted on a distal end of an over-tube.

14. The endoscopic system of claim 12, wherein the protrusion is disposed on a straight line interconnecting a center of the endoscope channel and a center of the treatment tool channel to one another.

15. The endoscopic system of claim 12, wherein the protrusion has a slope which protrudes radially inwardly by a distance that progressively varies from a proximal end toward the distal end of the endoscope channel.

16. The endoscopic system of claim 12, wherein when a distal end of the endoscope comes near an opening of the endoscope channel, the endoscope captures an image of the protrusion provided at the distal end of the endoscope channel and acquires an endoscopic image.

17. The endoscopic system of claim 16, the protrusion in the endoscopic image permits a user to know in which position the treatment tool will appear in the endoscopic image before the treatment tool projects from the treatment tool exit hole through the treatment tool channel and appears in the endoscopic image.

18. The endoscopic system of claim 12, wherein gaps are formed between the endoscope and an inner circumferential surface of the endoscope channel except for the protrusion so that a body fluid that being entered the endoscope channel tends to flow through the gaps rather than staying in the endoscope channel so that the endoscope channel is kept in a state that makes the protrusion visually recognizable.
